(19) United States
(12) Patent Application Publication
(10) Pub. No.: US 2020/0206446 A1
(43) Pub. Date: Jul. 2, 2020

(54) Oro-Nasal Ventilation Face Mask

(71) Applicant: Respitore, LLC, Cardiff By The Sea, CA (US)

(72) Inventor: Ian Blaxland, Cardiff By The Sea, CA (US)

(21) Appl. No.: 16/729,265

(22) Filed: Dec. 27, 2019

Related U.S. Application Data
(60) Provisional application No. 62/785,689, filed on Dec. 27, 2018.

(51) Int. Cl. A61M 16/06

(52) U.S. Cl. CPC A61M 16/06; A61M 16/0683; A61M 2210/0625; A61M 2210/0618; A61M 16/0622

(57) ABSTRACT

An oro-nasal ventilation face mask worn by a patient includes a first chamber receiving airflow from an air source, a second chamber in fluid communication with the first chamber, a restrictive element positioned between the first chamber and the second chamber, and a headgear securing the first chamber and the second chamber to the patient. The second chamber includes a vent. Air flows between the first and second chambers through the restrictive element. When the first and second chamber are secured to the patient, the first chamber has a first air pressure, the second chamber has a second air pressure, and the first air pressure is greater than the second air pressure.

### CROSS-REFERENCE TO RELATED APPLICATIONS

This application incorporates by reference and claims the benefit of priority to U.S. Provisional Patent Application No. 62/785,689 filed Dec. 27, 2019.

### BACKGROUND OF THE INVENTION

The present subject matter relates generally to face masks. More specifically, the present subject matter relates to face masks for oro-nasal ventilation that include a first chamber and a second chamber, with one or more inter-chamber connections providing a pressure differential between the two chambers.

Many currently available oro-nasal ventilation face masks provide air to the patient equally across both the nasal and oral passages, providing an equilibrium in pressure at the respiratory tract opening. During periods of apnea and at the end of expiration when respiratory flow is zero, air within the oro-nasal cavity remains stagnant even if both the nasal and oral passages are open. Further, this constant and equal pressure provided to the nasal and oral passages simultaneously is inefficient for patients that inhale primarily through one passage. The user or treatment provider is unable to customize the air flow and air pressure to address this issue.

Accordingly, a need exists for an oro-nasal ventilation face mask that provides separate chambers having a pressure differential therebetween, allowing the user or treatment provider to assign a customized pressure and flow profile to each chamber that more effectively meets the patient's needs.

### BRIEF SUMMARY OF THE INVENTION

To meet the needs described above and others, the present disclosure provides an oro-nasal ventilation face mask including a first chamber and a second chamber connected through a restrictive element. Each chamber is fully enclosed and sealed against the patient's face during use. The separation enables different air pressures to be maintained at the patient's nasal passage and oral passage in order to promote breathing. The face mask is secured to headgear which maintains the positioning of the face mask on the patient during sleep.

In one embodiment, the first chamber is an oral chamber and the second chamber is a nasal chamber. The oral chamber receives airflow from an inflow tube, and air is provided to the nasal chamber through the restrictive element. A constant flow of air through the resistive element occurs by placing at least one vent, or diffuser, in the second nasal chamber in this embodiment.

A front frame is positioned on an external front portion of the oral chamber. The front plate includes slots on opposite sides to receive and hold straps of the headgear. The front plate also includes an opening for receiving the inflow tube. A second opening within the front portion of the oral chamber receives an internal flange on the opening of the front plate, providing an airtight seal where the inflow tube engages with the face mask.

The restrictive element positioned between the oral chamber and the nasal chamber allows air to flow into the nasal chamber. The restrictive element may be, for example, a restrictive orifice, a low-pressure valve, a venturi element, a laminar flow resistor, one or more resistive tubes, or another suitable apparatus, and may include baffles or other diffusive elements to guide the flow of air and reduce noise. The purpose of the restrictive element is to create a pressure differential between the oral chamber and the nasal chamber, enabling the air pressure in the oral chamber to be greater than the air pressure in the nasal chamber. The pressure differential in this embodiment allows air to flow into the patient's mouth, through the patient's oro-nasal cavity, and out through the patient's nose. In other embodiments, the air pressure in the nasal chamber may be greater than the air pressure in the oral chamber, causing air to flow freely into the patient's nose, through the patient's oro-nasal cavity, and out through the patient's mouth.

The oro-nasal ventilation face mask described herein may also include an anti-asphyxia safety valve in one or both chambers which allows air to flow into the mask when positive air pressure is not being provided by an air pump or positive pressure machine.

In one embodiment, the air flows into the mask into a first chamber chambers, and a vent or diffuser that is open to atmosphere is located in the second chamber. It is contemplated that the inflow may be to either the nasal or oral chamber, with the corresponding vent or diffuser in the other chamber.

In a first embodiment, the first chamber may be an oral chamber and the second chamber may be a nasal chamber. In a second embodiment, the first chamber is a nasal chamber and the second chamber is an oral chamber.

Each of the first chamber and the second chamber may include a cushion along a sealing edge of each of the first chamber and the second chamber that, in combination with the headgear, assists in sealing the chamber against the user.

One advantage to the invention is that the bias flow significantly reduces the amount of physiological dead space within the respiratory tract.

A further benefit of the invention is that the bias flow lowers the metabolic demand attributed to respiratory muscles for patients with various types of pulmonary disorders.

An advantage to the invention is that by creating a pressure differential between the two chambers, the flow of air can be tailored to suit each patient's needs.

Another advantage to the invention is that creating a pressure differential between the nasal passages and oral passage will allow air to flow freely into the patient's oro-nasal cavity through the cavity opening within the higher pressure chamber.

Another advantage of the invention is that creating a pressure differential between the two chambers can improve upper airway patency during events of the patients mouth opening.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a front, perspective view of a face mask and headgear of the present application.

FIG. 2 is an enlarged view of the face mask of FIG. 1.

FIG. 3 is a block diagram of airflow through the components of the face mask of FIG. 1.

FIG. 4 is a rear, perspective view from below of the face mask of FIG. 1.

FIG. 5 is an exploded, side, perspective view of the face mask of FIG. 1.

FIG. 6 is a front perspective view of the face mask of FIG. 1.

FIG. 7 is a front elevational view of the face mask of FIG. 1.

FIG. 8 is a side elevational view of the face mask of FIG. 1.

FIG. 9 is a plan view of the face mask of FIG. 1.

FIG. 10 is a front elevational view of the face mask and headgear of FIG. 1.

FIG. 11 is a side elevational view of the face mask and headgear of FIG. 1.

FIG. 12 is a perspective view of an alternative embodiment of the face mask of FIG. 1.

### DETAILED DESCRIPTION OF THE INVENTION

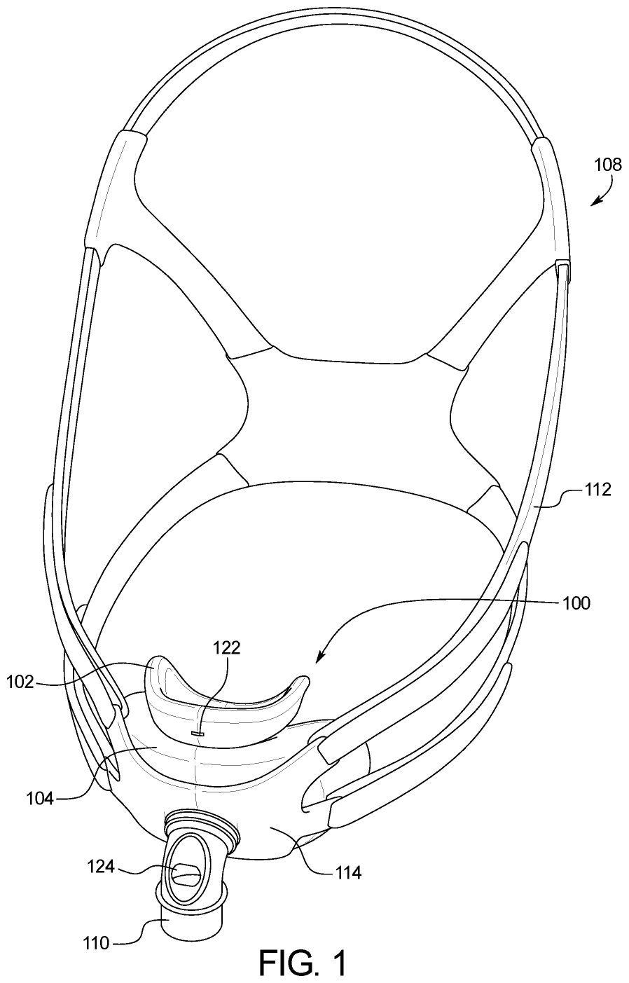

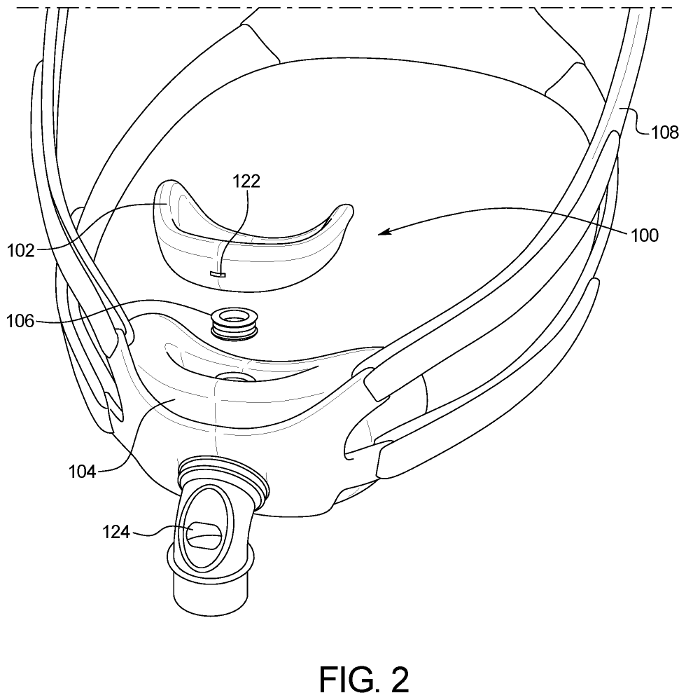

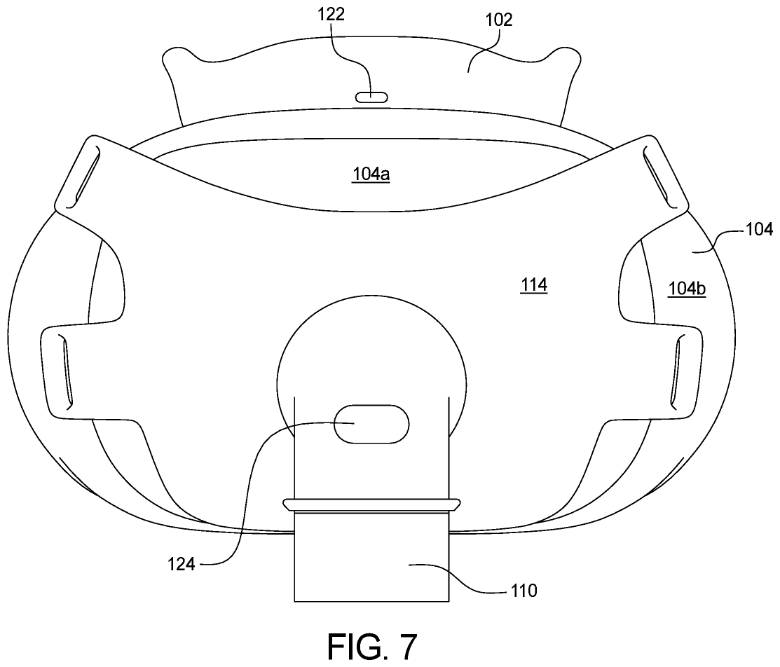

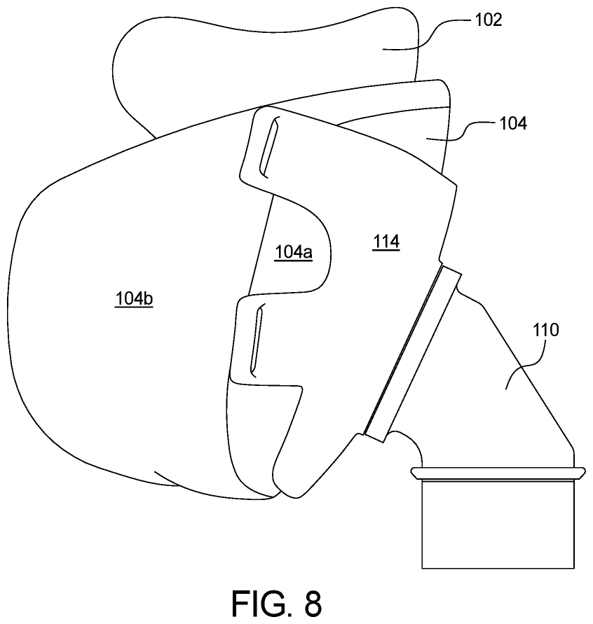

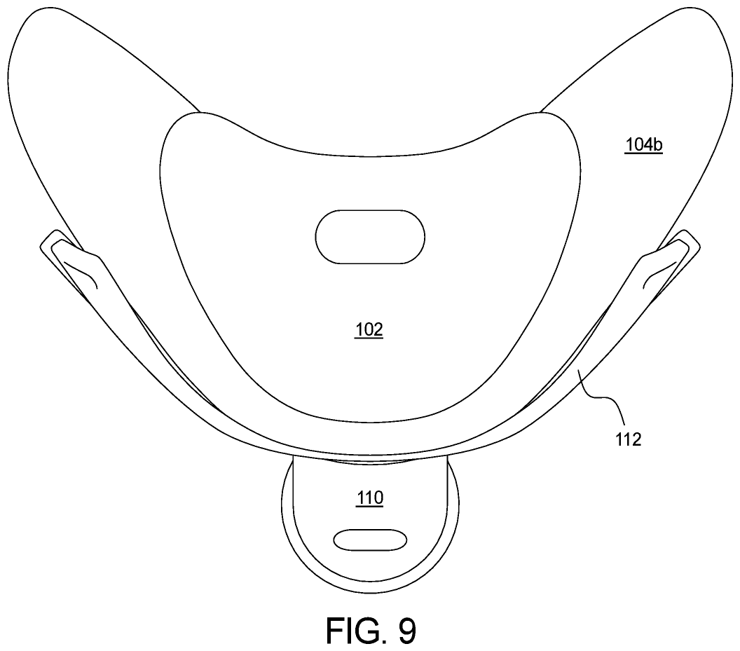

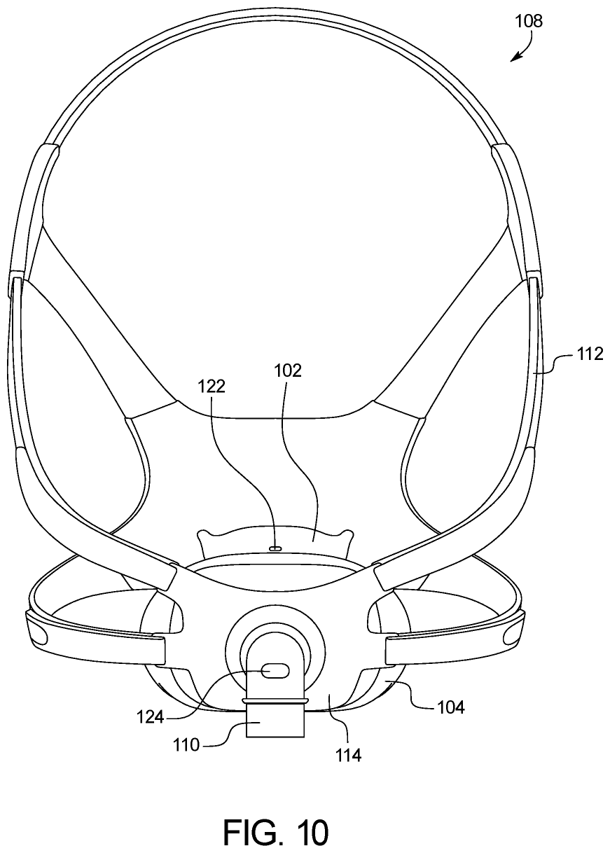

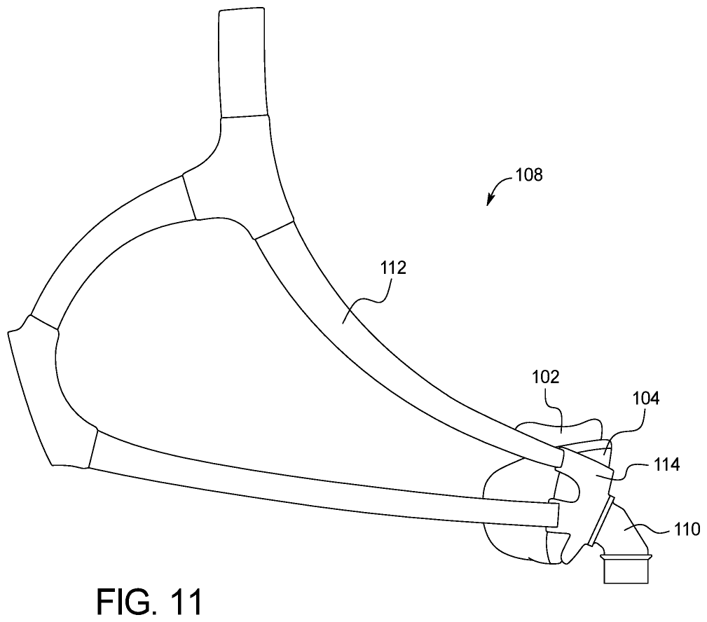

FIGS. 1-11 illustrate an example of an oro-nasal ventilation face mask 100. As shown in FIG. 1, the oro-nasal ventilation face mask 100 includes a nasal chamber 102 and an oral chamber 104 having a restrictive element 106 positioned therebetween. Each of the nasal chamber 102 and the oral chamber 104 is fully enclosed and sealed against the user's face during use, aside from the connection at the restrictive element. This separation allows for different air pressures to be maintained in the nasal and oral chambers 102, 104 while simultaneously utilizing a single air source. The pressure differential promotes breathing within the patient. In the illustrated embodiment, the face mask 100 is secured to headgear 108 which maintains the positioning of the face mask 100 on the patient during sleep.

In the embodiment illustrated in FIG. 1, an inflow tube 110 providing air from an air source to the face mask 100 connects to the oral chamber 104. The inflow tube 110 may be of any design or configuration, such as, for example, a tube providing continuous positive airway pressure from a positive airway pressure ventilator or respiratory machine (CPAP/BiPAP). Typically, the air flowing into the oral chamber 104 is under a prescribed pressure.

A restrictive element 106 positioned between the oral chamber 104 and the nasal chamber 102 allows air to flow into the nasal chamber 102, while a vent 122 in the nasal chamber 102 is open to the ambient environment and allows air to escape. The restrictive element 106 and vent 122 operate in conjunction to maintain an air pressure within the nasal chamber lower than an air pressure in the oral chamber 104. The restrictive element 106 may be, for example, a restrictive orifice, a low-pressure valve, a venturi element, a laminar flow resistor, one or more of resistive tubes, or another suitable apparatus, and may include baffles or other diffusive elements to guide the flow of air and reduce noise. The pressure drop effectuated by airflow through the restrictive element 106 depends on the length, the cross-sectional area, and the surface area roughness of the restrictive element 106. These aspects may vary as desired.

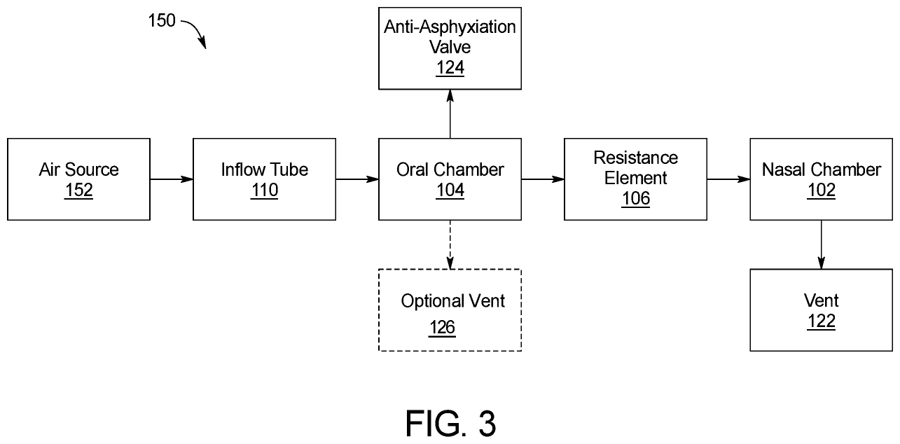

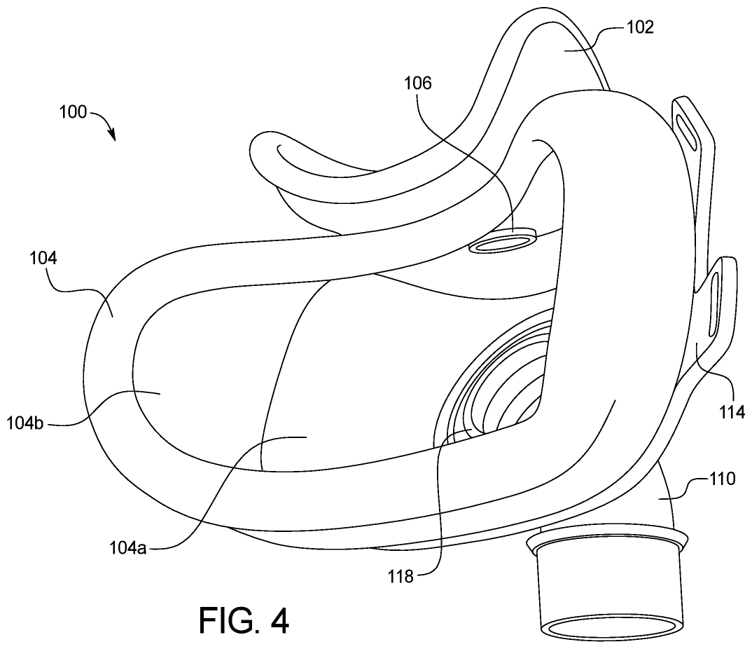

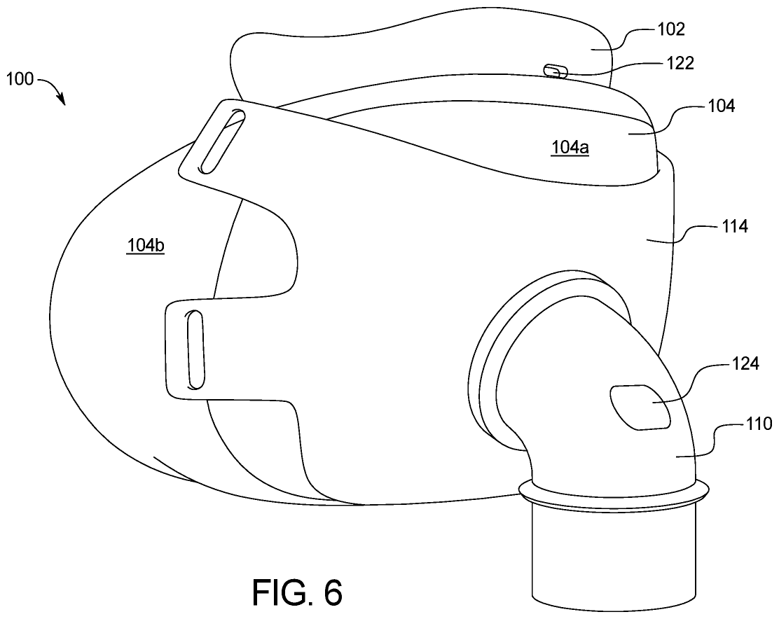

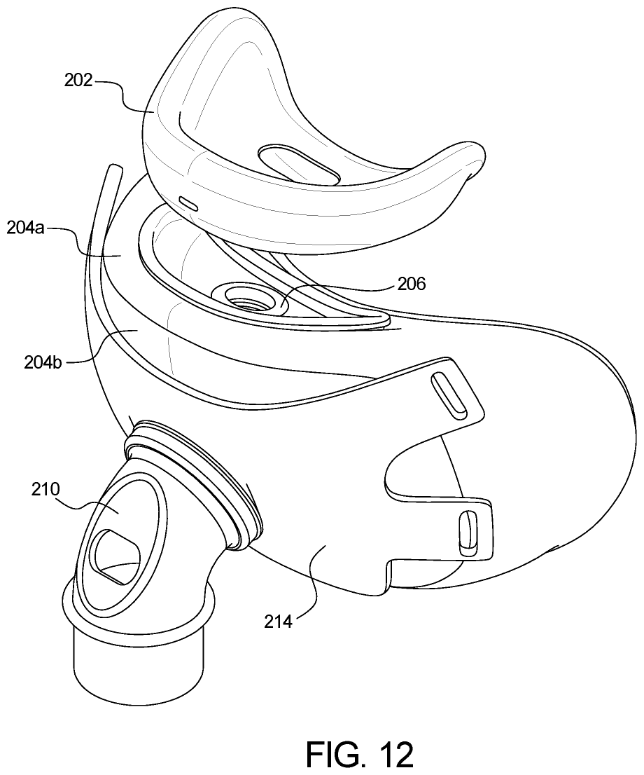

In the embodiment illustrated in FIGS. 4, the restrictive element 106 is a component separate and apart from the chambers 102, 104. In other embodiments, the restrictive element may be formed integrally with either of the nasal chamber 102 or the oral chamber 104. For example, in the alternative embodiment of the face mask 200 illustrated in FIG. 12, the restrictive element 206 is formed integrally with the body portion 204a of the oral chamber 204. In other embodiments, the restrictive element may include multiple components that are formed integrally with both of the oral and nasal chambers 102, 104. The restrictive element 106 may have any size or shape as desired, with an opening having a cross-sectional area ranging between about 20 mm² to about 500 mm² in a preferred embodiment, and ranging from about 70 mm² to about 315 mm² in a more preferred embodiment. The vent 122 may be any shape or size, ranging from multiple small orifices to a singular shape as shown in FIG. 6. Additional vents 126 (see FIG. 3) may optionally be provided on either of the nasal or oral chamber 102, 104, as desired.

In the embodiment described in FIGS. 1-11, the air pressure in the oral chamber 104 is greater than the air pressure in the nasal chamber 102. The pressure differential in this embodiment allows air to flow freely into the patient's mouth, through the patient's oro-nasal cavity, and out through the patient's nose. In another embodiment, the inflow tube 110 connects to the nasal chamber 102 and a vent is provided on the oral chamber 104. This embodiment maintains a greater air pressure in the nasal chamber 102 and a lower air pressure in the oral chamber 104, allowing air to flow freely into the patient's nose, through the patient's oro-nasal cavity, and out through the patient's mouth.

Referring again to FIG. 1, an anti-asphyxiation valves 124 is provided on the inflow tube 110. The anti-asphyxiation valve 124 may be provided on the inflow tube 110 or the chamber directly receiving air from the inflow tube 110 and therefore having a higher air pressure. The anti-asphyxiation valve 124 prevents suffocation in the event that positive incoming air pressure from the air source ceases. The opening of the anti-asphyxiation valve 124 is triggered by the lack of positive air pressure and allows atmospheric air to fill the adjacent chamber, enabling the patient to breathe through his or her mouth without impediment.

FIG. 3 illustrates the air fluid path 150 through the components of the face mask 100. Air is provided by an air source 152, such as a continuous or bilevel positive airway pressure device. Air is routed from the air source 152 through tubing 110 to an oral chamber 104. Ambient air may enter the oral chamber 104 through an anti-asphyxiation valve 124 if the airflow from the air source 152 stops. The oral chamber 104 may also include optional air vents 154. Air travels from the oral chamber 104 through the restrictive element 106 and into the nasal chamber 102. A portion of the air in the nasal chamber 102 escapes the face mask 100 through a vent 122, allowing the air pressure in the nasal chamber 102 to be less than an air pressure in the oral chamber 106.

Referring to FIGS. 1, 2, 10, and 11, the headgear 108 includes straps that attach to opposing sides of a front plate 114 of the face mask 100. The patient may adjust the straps to fit the contours of the patient's head. In some embodiments, the head straps may include a quick release feature so the user may easily disconnect the headgear 108 from the face mask 100.

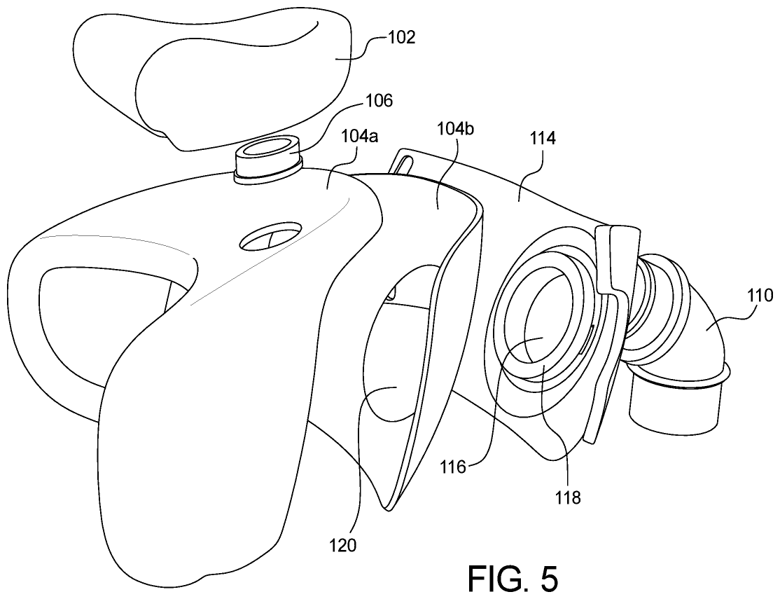

Referring to FIGS. 4-8, the oral chamber 104 includes a body portion 104a and a front portion 104b that couple together to form an airtight seal at its juncture. Shown best in FIG. 5, the front plate 114 is positioned on an outer surface of the front portion 104b of the oral chamber 104, and includes an opening 116 onto which the inflow tube 110 is secured. An internal flange 118 on the opening 116 of the front plate 114 is received by an opening 120 on the front portion 104a of the oral chamber 104, forming an airtight seal. During use, the front plate 114 and the front portion 104b of the oral chamber 104 are secured together.

In some embodiments, each chamber 102, 104 has a cushion around its edge which improve the seal and prevent air leaks from the chamber. The cushion may comprise silicone or another material with similar properties (e.g., resilient, cushioned, comfortable against the user's skin, etc.).

It should also be noted that while particular combinations of features have been described, the scope of the present invention is not limited to the particular combinations presented herein, but instead extends to encompass any combination of features disclosed. Various changes and modifications to the presently preferred embodiments described herein will be apparent to those skilled in the art. Such changes and modifications may be made without departing from the spirit and scope of the present invention and without diminishing its attendant advantages.

I claim:

1. An oro-nasal ventilation face mask worn by a patient comprising:
a first chamber receiving airflow from an air source;
a second chamber in fluid communication with the first chamber, wherein the second chamber includes a vent;
a restrictive element positioned between the first chamber and the second chamber, wherein air flows between the first and second chambers through the restrictive element; and
a headgear releasably securing the first chamber and the second chamber to the patient;
wherein, when the first and second chamber are secured to the patient, the first chamber has a first air pressure, the second chamber has a second air pressure, and the first air pressure is greater than the second air pressure.

2. The oro-nasal ventilation face mask of claim 1, wherein the first chamber is an oral chamber and the second chamber is a nasal chamber.

3. The oro-nasal ventilation face mask of claim 1, wherein the restrictive element is integral with one of the first and second chambers.

4. The oro-nasal ventilation face mask of claim 1, wherein the restrictive element comprises one or more restrictive orifices.

5. The oro-nasal ventilation face mask of claim 1, wherein the restrictive element comprises a venturi element.

6. The oro-nasal ventilation face mask of claim 1, wherein the restrictive element comprises one or more resistive tubes.

7. The oro-nasal ventilation face mask of claim 1, wherein the restrictive element comprises one or more diffusive elements.

8. The oro-nasal ventilation face mask of claim 1, wherein the first chamber includes a vent is open to an ambient environment.

9. The oro-nasal ventilation face mask of claim 1, wherein one of the first chamber, the second chamber, and the restrictive element includes an anti-asphyxiation valve that allows air from an ambient environment to enter when airflow into the first chamber stops.

10. The oro-nasal ventilation face mask of claim 1, wherein the restrictive element includes an opening having a cross-sectional area ranging between about 20 mm² to about 500 mm².

11. The oro-nasal ventilation face mask of claim 1, wherein the restrictive element includes an opening having a cross-sectional area ranging between about 70 mm² to about 315 mm².

12. The oro-nasal ventilation face mask of claim 1, further comprising a front frame positioned against the first chamber, wherein the headgear attaches to opposing sides of the front frame.

13. The oro-nasal ventilation face mask of claim 12, wherein the front frame is positioned against a front surface of the first chamber.
